(19) United States
(12) Patent Application Publication
(10) Pub. No.: US 2024/0324628 A1
(43) Pub. Date: Oct. 3, 2024

(54) Pet Treat Pocket

(71) Applicant: Shauna Angelidis, Houston, TX (US)

(72) Inventor: Shauna Angelidis, Houston, TX (US)

(21) Appl. No.: 18/738,401

(22) Filed: Jun. 10, 2024

Related U.S. Application Data
(63) Continuation-in-part of application No. 17/543,939, filed on Dec. 7, 2021.
Application No. 17/543,939 is a continuation-in-part of application No. 17/011,045, filed on Sep. 3, 2020, now abandoned.
(60) Provisional application No. 62/940,342, filed on Nov. 26, 2019.

(51) Int. Cl. A23K 40/30; A23K 20/147; A23K 20/163; A23K 20/28; A23K 50/42; A61K 9/00

(52) U.S. Cl. CPC A23K 40/30; A23K 20/147; A23K 20/163; A23K 20/28; A23K 50/42; A61K 9/0056

(57) ABSTRACT

A pet treat pocket that includes a first pocket disposed on a top surface of a flexible member and a second pocket disposed on a lower surface of the flexible member. The pockets each define an interior volume sized to encapsulate a pill or liquid medication, respectively. An exterior length of each pocket is less than a length of the flexible member, and an exterior width of each pocket is less than a width of the flexible member such that the flexible member extends beyond the exterior dimensions of each pocket forming a lip surrounding a circumference of each pocket. The flexible member and the pockets are both composed of an ingestible and pet-safe material. The ingestible and pet-safe material may adhere to itself such that the flexible material may be molded to completely envelope the pockets and maintain a sealed configuration.

### CROSS-REFERENCE TO RELATED APPLICATIONS

This application is a continuation-in-part and claims the benefit of U.S. patent application Ser. No. 17/543,939 filed on Dec. 7, 2021, which claims benefit as a continuation in part to U.S. patent application Ser. No. 17/011,045 filed on Sep. 3, 2020, which in turn claims the benefit of U.S. Provisional Application No. 62/940,342 filed on Nov. 26, 2019. The above identified patent applications are herein incorporated by reference in their entirety to provide continuity of disclosure.

### BACKGROUND OF THE INVENTION

The present invention relates to pet treats and medications. More particularly, the present invention provides for a pet-safe ingestible material which includes a pocket. The present invention may be offered by itself as a healthy treat to a pet, or a pill may be inserted into the pocket and the material hand molded to encapsulate the pill within the pocket. The pet-safe ingestible material may block or mask odors from the pill such that a pet in unable to detect the presence of the pill within the pocket.

Many people have pets who require medication for maintenance or improvement of their health or well-being. However, such medications are often challenging to feed to these pets. Some pets refuse direct ingestion of medications. In such circumstances, it may be difficult or impossible to administer the medications. Some pets are able to detect medications by smell even when hidden within food and may eat around the medication.

Devices have been disclosed in the known art that relate to pet treats and medications. These include devices that have been patented and disclosed in patent application publications. However, the devices in the known art have several drawbacks. Some devices include an opening in which a pill may be placed. However, these devices may not secure the pill and the pill may fall out. Other devices may not prevent the pet from detecting the presence of the pill, either by sight or smell. Other devices may be ingestible but contain chemicals and ingredients that are harsh on the pet's digestive system and do not provide much nutritional value.

The present invention substantially diverges in design elements from the known art and consequently it is clear that there is a need in the art for an improvement to existing pet treat and medication devices. In this regard the present invention substantially fulfills these needs.

### SUMMARY OF THE INVENTION

In view of the foregoing disadvantages inherent in the known types of pet treat and medication devices now present in the art, the present invention provides a pet-safe ingestible material which includes a pocket. The present invention may be offered by itself as a healthy treat to a pet, or a pill may be inserted into the pocket and the material hand molded to encapsulate the pill within the pocket. The pet-safe ingestible material may block or mask odors from the pill such that a pet in unable to detect the presence of the pill within the pocket. The present pet treat pocket comprises a flexible member including a pocket disposed on a top surface of the flexible member. The pocket defines an interior volume and the interior volume is sized to encapsulate a pill. An exterior length of the pocket is less than a length of the flexible member, and an exterior width of the pocket is less than a width of the flexible member such that the flexible member extends beyond the exterior dimensions of the pocket forming a lip surrounding a circumference of the pocket. The flexible member and the pocket are both composed of an ingestible and pet-safe material. The ingestible and pet-safe material may adhere to itself such that the flexible material may be molded to completely envelope the pocket and maintain a sealed configuration.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of an embodiment of the pet treat pocket being used to receive a pill.

FIG. 2 shows a front perspective view of a row of seven pet treat pockets in an embodiment of the pet treat pocket.

FIG. 3 shows a front perspective view of an embodiment of the pet treat pocket illustrating how the pet treat pocket may be folded to enrobe a pill.

FIG. 4 shows a chart of a method of use of an embodiment of the pet treat pocket.

FIG. 5 shows a lower perspective view of an alternate embodiment of the pet treat pocket.

FIG. 6 shows a cross-sectional view of an alternate embodiment of the pet treat pocket.

FIG. 7 shows a perspective view of an alternate embodiment of the pet treat pocket in use.

### DETAILED DESCRIPTION OF THE INVENTION

Reference is made herein to the attached drawings. Like reference numerals are used throughout the drawings to depict like or similar elements of the pet treat pocket. For the purposes of presenting a brief and clear description of the present invention, a preferred embodiment will be discussed as used for the pet treat pocket. The figures are intended for representative purposes only and should not be considered to be limiting in any respect.

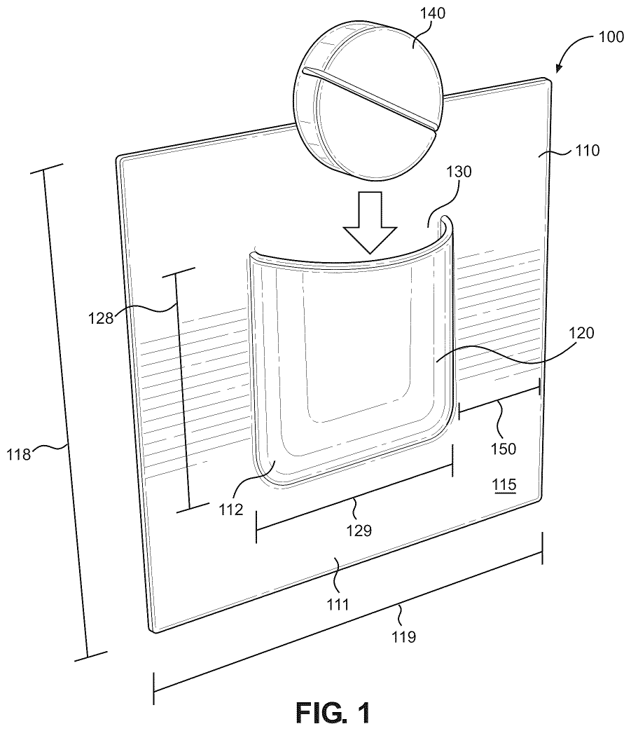

Referring now to FIG. 1, there is shown a front perspective view of an embodiment of the pet treat pocket being used to receive a pill. The pet treat pocket 100 comprises a flexible member 110 including a pocket 120 disposed on a top surface 115 of the flexible member 110. In some embodiments, the flexible member 110 and the pocket 120 are composed of a single layer of a material. In such embodiments, production and manufacturing costs are cut to a minimum and the likelihood of the flexible member 110 detaching is eliminated. In other embodiments, the flexible member 110 is a first layer 111 and the pocket 120 is a second layer 112 disposed on a top surface 115 of the flexible member 110. Such embodiments provide easy manufacturing and assembly of the pet treat pocket 100.

The pocket 120 defines an interior volume 130. The interior volume 130 is sized to encapsulate at least one pill 140. As one object of the present invention is to hide and mask the appearance and odors from the pill 140, one of ordinary skill in the art will understand the interior volume 130 dimensions may vary from one embodiment to another based on the size of the pill 140. In the shown embodiment, the pocket 120 is connected to the flexible member 110 on three of four sides. The fourth, open side, enables a user to slide the pill 140 into the pocket 120. In the shown embodiment, the entirety of the fourth side is open, however, in other embodiments, only a portion of the fourth side is open with the rest of the fourth side connected to the flexible member 110. In the shown embodiment, the pocket 120 comprises an arcuate shape in order to facilitate easier insertion of the pill 140 into the pocket 120. In such embodiments, a depth of the pocket 120 at a central portion of the pocket 120 is greater than a depth at each lateral side of the pocket 120. As such, the pocket 120 is dimensioned to receive pills 140 of various common shapes and structures.

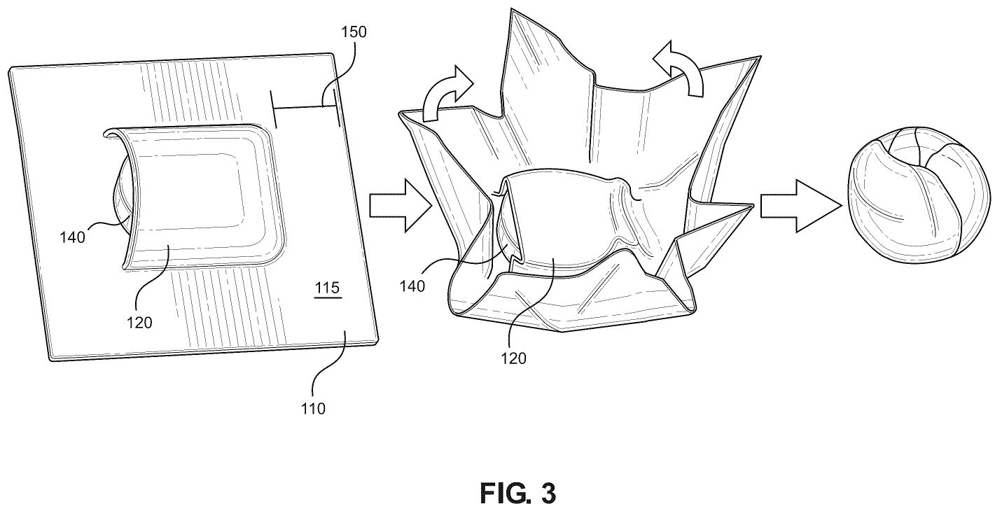

An exterior pocket length 128 is less than a flexible member length 118, and an exterior pocket width 129 is less than a flexible member width 119 such that the flexible member 110 extends beyond the exterior dimensions of the pocket 120, thereby forming a lip 150. In the shown embodiment, the lip 150 surrounds a circumference of the pocket 120. In the preferred embodiment, the lip 150 is sized to completely enrobe an entirety of the pocket 120 when the flexible member 110 is molded over the pocket 120 (as shown in FIG. 3).

The flexible member 110 and the pocket 120 are both composed of an ingestible and pet-safe material. In the preferred embodiment, the ingestible and pet-safe material is organic and is palatably flavored in order to minimize inorganic compounds ingested and to promote a pet to eat the pet treat pocket by the nature of its flavor. In some such embodiments, the ingestible and pet-safe material comprises a flavoring additive therein, wherein the flavoring additive may comprise a variety of possible flavors desirable and palatable to an animal, such as, but not limited to beef, chicken, fish, or the like. In some embodiments, the ingestible and pet-safe material is a thin gelatin-based material. An object of the present invention is to enrobe a pill 140 in the pet treat pocket 100 and the thin gelatin-based material enables the invention to be easily folded and molded. In the preferred embodiment, the ingestible and pet-safe material is malleable by hand thereby enabling a user to manipulate and mold the material without any additional tools.

In a preferred formulation, the ingestible and pet-safe material comprises montmorillonite clay and a binding material, such as gelatin. The montmorillonite clay provides significant health benefits to the pet in that it offers 67 different minerals including: calcium, iron, magnesium, potassium, and the like. The montmorillonite clay also has a detoxifying agent which eliminates toxins from the body. It has proven to aid skin and ear allergies, digestive disturbances, thyroid, adrenal and pancreatic disorders, seizures, kidney disease and liver issues. It also relieves arthritic pain, skin infections, allergic reactions, vomit, and diarrhea. The clay may also draw out toxins, allergens, parasites, and bacteria while detoxifying the intestinal tract of the pet. Relatedly, calcium bentonite has been used in similar clays as an anti-caking agent which is added to the formulation to absorb excess moisture and aids in preventing foods from clumping. The present invention does not utilize such anti-caking agents, and thus in the preferred embodiments, the ingestible and pet-safe material does not compromise calcium bentonite.

The ingestible and pet-safe material is configured to adhere to itself upon the application of pressure. As mentioned previously, an object of the present invention is to enrobe and encapsulate a pill 140 within the invention. As the ingestible and pet-safe material may adhere to itself, upon the material being folded and molded into a desired geometry, the geometry is maintained as a function of this self-adherence. Of particular note is the fact that by virtue of the pocket 120 being disposed on the top surface 115 of the flexible member 110, in various embodiments, the present device provides a double enrobing of the pill 140; the first from the pocket 120 itself, and the second from wrapping the lip 150 and the remaining portions of the flexible member 110 around the pill 140. In this manner, when the flexible member 110 is folded about the pocket 120, a double wall is defined between an interior volume of the pocket 120 and the surrounding area. A first wall of the double wall comprises an exterior wall of the pocket 120 and the second wall of the double wall comprises the lip 150 folded over the exterior wall of the pocket 120. In this manner, the interior volume is more securely masked by several layers of material, thereby disguising the pill 140 within the interior volume.

Another object of the present invention is to hide, mask, block, or otherwise prevent the pet from detecting the presence of the pill 140 with the pet treat pocket 100. Some pets have difficulty taking pills, typically by virtue of the smell and taste of the pill 140. Thus, one object of the present invention is to eliminate the odor such that the pet will ingest the pet treat pocket without problem. In one embodiment, upon the pill 140 being encapsulated by the pocket 120, the ingestible and pet-safe material blocks odors emanating from the pill 140 from escaping the pocket 120. In this manner, a pet will have a harder time detecting the presence of the pill 140. In another embodiment, upon the pill 140 being encapsulated by the pocket 120, the ingestible and pet-safe material masks odors emanating from the pill 140 from escaping the pocket 120, such as by providing a stronger scent that covers the odors emanating from the pill 140. In this manner, the pet also has a harder time detecting the presence of the pill 140 as the alternate odors may cover the odors emanating from the pill 140. Additionally, by virtue of the double enrobing of the pill 140, odors emanating from the pill 140 are similarly masked or blocked.

A soft inner hygoscopic (glycerin based with multiple hydroxyl groups) humectant to attract and retain fluids. This creates an inner sweet tasting softness that allows for easy insert of a tablet and prevention of the migration of moisture to the outer casing risking losing its palatable crunchiness that pets favor. In addition, it keeps the texture soft and chewable as the treats do not dry out, extends shelf life by inhibiting the growth of microbes such as mold and bacteria, which thrive in dry conditions. This extends the shelf life of the treats without the need for excessive preservatives. In addition, it allows for quality and consistency preventing the product from becoming brittle and hard, ensuring a consistent texture that pets enjoy.

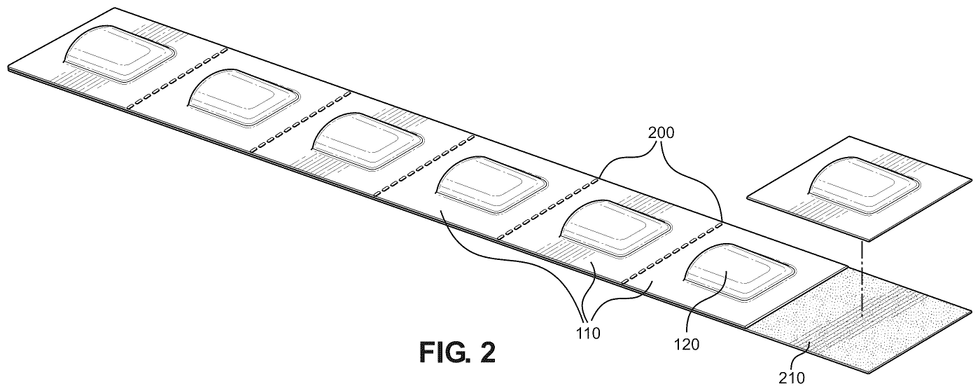

Referring now to FIG. 2, there is shown a front perspective view of a row of seven pet treat pockets in an embodiment of the pet treat pocket. In the shown embodiment, a plurality of pockets 120 are disposed on the flexible member 110. Further, in the shown embodiment, seven (7) pockets 120 are disposed on the flexible member 110, such that a user may plan to utilize a single pocket 120 for each day of the week. As some pets require multiple pills daily, the pocket 120 may be sized to accommodate multiple pills at once. In this manner, a single pocket 120 may be filled with desired medication and detached from the flexible member 110 each day of the week. In the shown embodiment, at least one perforation 200 separates at least one of the plurality of pockets 120 from a neighboring pocket 120 of the plurality of pockets 120. As pills may be of different sizes, or multiple doses of pills required per day, in such an embodiment, a user may selectively choose the number of pockets 120 required and separate those pockets 120 for use throughout the day/week/desired timeframe.

In the shown embodiment, a backing material 210 is disposed along a rear surface of the flexible member 110. The backing material 210 is removably secured to the rear surface of the flexible member 110 such that when a user desires to utilize a given pocket 120, they may remove the backing material 210 to expose the rear surface of the flexible material 110. The backing material 210 provides the benefit of reducing the amount of exposure that the flexible material 110 has to the environment, and thus protects the flexible material 110 from becoming dirty, contaminated, or the like. Additionally, such reduction in exposure to the environment may aid in preserving the self-adhesive nature of the flexible member 110.

Referring now to FIG. 3, there is shown a front perspective view of an embodiment of the pet treat pocket illustrating how the pet treat pocket may be folded to enrobe a pill. In the shown embodiment, a pill 140 may be inserted into a pocket 120 disposed on a top surface 115 of a flexible member 110. As the pocket 120 is dimensioned smaller than the flexible member 110, a lip 150 is formed and defined. The lip 150 may be physically manipulated to enrobe and surround the pocket 120 by folding the lip 150 up, over, and around the pocket 120, thereby encasing and securing the pill 140 within the pocket 120. In one embodiment, the lip 150 extends at least as wide as half the pocket width plus the pocket depth, such that when opposing sides of the lip 150 are folded over the pocket 120, the lip 150 fully encases the pocket 120. In some embodiments, the flexible member 110, and therefore the lip 150, is configured to adhere to itself. By nature of such self-adherence, once the pill 140 and pocket 120 are molded into a desired shape, such shape is held in place. In this manner, once the user lets go of the invention, for example by offering it to a pet, the flexible member 110 and lip 150 will not unravel and reveal the presence of the pill 140.

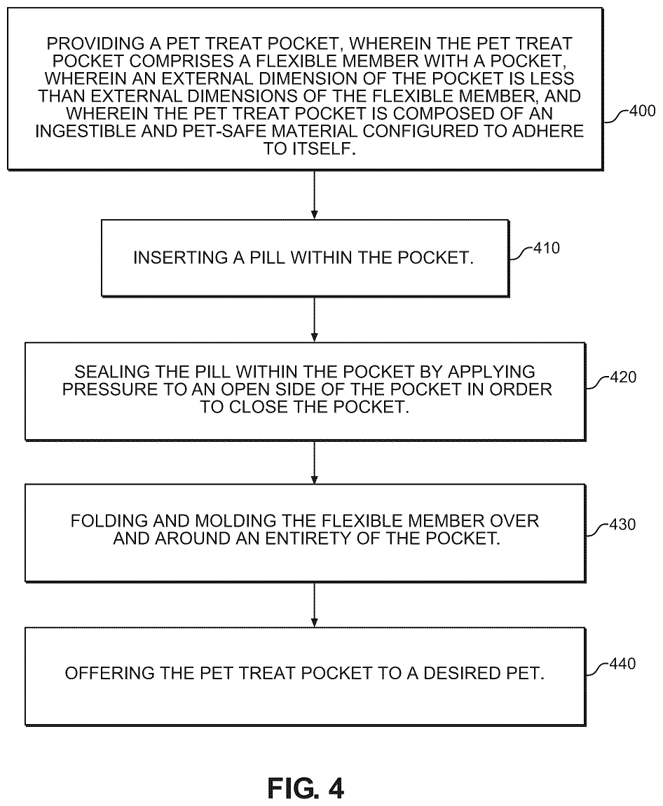

Referring now to FIG. 4, there is shown a chart of a method of use of an embodiment of the pet treat pocket. An object of the present invention is to provide a healthy treat for a pet, as well as to provide a mechanism through which a pill may be hidden and offered to the pet. As the invention may be a stand-alone treat, any mention of a pill and related steps, are optional in some embodiments, as the pet treat pocket may be offered without a pill. A method of use comprises the steps of first providing a pet treat pocket, wherein the pet treat pocket comprises a flexible member with a pocket, wherein an external dimension of the pocket is less than external dimensions of the flexible member, and wherein the pet treat pocket is composed of an ingestible and pet-safe material configured to adhere to itself 400. A pill is inserted into the pocket 410 and then the pill is sealed within the pocket by applying pressure to an open side of the pocket in order to close the pocket 420. The pressure applied causes the open side of the pocket to adhere to the flexible member by virtue of the self-adhesive properties of the ingestible and pet-safe material. Folding and molding the flexible member over and around an entirety of the pocket 430 ensures that the pill is contained therein, and any visual or other sensory cues as to the presence of the pill are masked, blocked, or similarly prevented. The final step is the offering of the pet treat pocket to a desired pet 440 for this ingestion and consumption.

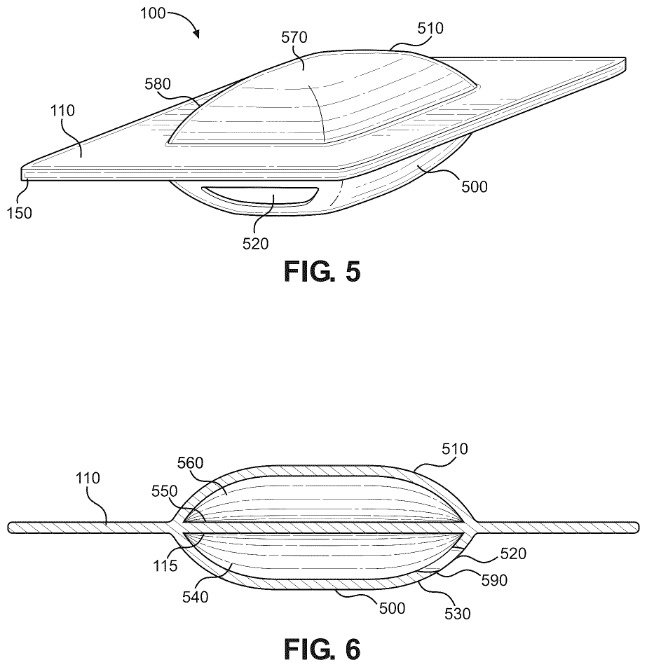

Referring now to FIG. 5, there is shown a lower perspective view of an alternate embodiment of the pet treat pocket. The shown embodiment comprises an alternate structure comprising a duality of pockets, however the prior descriptions regarding material, dimensions, and the like are contemplated to be incorporated into the following embodiment. In the shown embodiment, the pet treat pocket 100 comprises a dual pocket structure having a first pocket 500 centrally disposed on a top surface of the flexible member 110, wherein the first pocket 500 comprises an opening 520 through an upper edge (as shown in FIG. 6, 530) thereof providing access to a first interior volume (as shown in FIG. 6, 540). Directly opposite the first pocket 500 is centrally disposed a second pocket 510 disposed on a lower surface (as shown in FIG. 6, 550) of the flexible member 110, wherein the second pocket 500 comprises an upper wall 570 opposite the flexible member 110 and a plurality of sidewalls 580 fully enclosing a second interior volume (as shown in FIG. 6, 560). The second pocket 510 is contemplated to contain a fluid medication injected therein via a syringe, such that the pet treat pocket 100 includes a single means of medicating an animal with a solid medication, a liquid medication, or a combination thereof. As such, in some embodiments, the second pocket 510 further comprises an interior ingestible and pet safe gel coating to ensure that the second pocket 510 is fluid impermeable. In this manner, the fluid medication does not seep through the pet treat pocket 100 or into the first pocket 500 through the flexible member 110. As previously described herein, the first and second pockets 500, 510 are dimensioned such that a width and height of each of the first and second pockets 500, 510 is less than a corresponding length and width of the flexible member 110 defining a lip 150 therearound, the lip 150 configured to enrobe an entirety of one of the first and second pockets 500, 510 when folded therearound. The first and second pockets 500, 510 of the shown embodiment are similarly contemplated to comprise the dimensions of the previously described embodiment of FIGS. 1 and 2. Similarly, the flexible member 110 of the shown embodiment is also contemplated to be affixed to additional pet treat pockets 100 in an elongated strip separated by perforations extending perpendicularly across the elongated strip, wherein the opening 520 of each first pocket 500 is aligned substantially parallel to the lines of perforation. In some such embodiments, the elongated strips comprise a series of seven pet treat pockets 100 allowing a user to associate each pet treat pocket 100 with a particular day of the week to ensure that medication is provided to a pet as necessary.

Referring now to FIG. 6, there is shown a cross-sectional view of an alternate embodiment of the pet treat pocket. In the illustrated embodiment, the first pocket 500 is disposed on the top surface 115 of the flexible member 110, wherein the first pocket 500 is affixed to the flexible member 110 along at least three sides thereof. In the illustrated embodiment, the opening 520 is disposed through a fourth side of the first pocket 500, wherein the opening 520 provides access to the first interior volume 540. In the shown embodiment, the opening 520 is raised relative to the top surface 115 of the flexible member 110, such that the lower edge of the opening 520 is disposed at a greater height that a height of the flexible member 110 such that the size of the opening 520 is minimized to facilitate closure thereof by reducing the distance required to mold the material of the first pocket 500 to adhere to the flexible member 110. In this manner, a pill or inserted into the first interior volume 540 may be retained therein with minimal impact to the dimensions of the first interior volume 540. Similarly, in the shown embodiment, the upper edge 530 of the first pocket 500 comprises an arcuate protrusion 590 extending therefrom, wherein the arcuate protrusion 590 further minimizes the effort required to adhere the upper edge 530 of the first pocket 500 to the flexible member 110 to close the first pocket 500. In such embodiments, the arcuate protrusion 590 may be adhered to the lower edge of the opening 520.

The second pocket 510 is disposed on the lower surface 550 of the flexible member 110, wherein the second pocket 510 comprises a fully enclosed second interior volume 560 configured to retain a fluid therein. In this manner, the second pocket 510 is comprises a fluid impermeable material preventing the transmission of fluid therein through the flexible member 110 and the upper wall and the plurality of sidewalls of the second pocket 510. In the illustrated embodiment, the first and second pockets 500, 510 comprise an identical volume and cross-sectional area, such that the first and second pockets 500, 510 are mirrored on opposing sides of the flexible member 110. In this manner, a user may provide equivalent amounts of medication to a pet in either liquid or solid form.

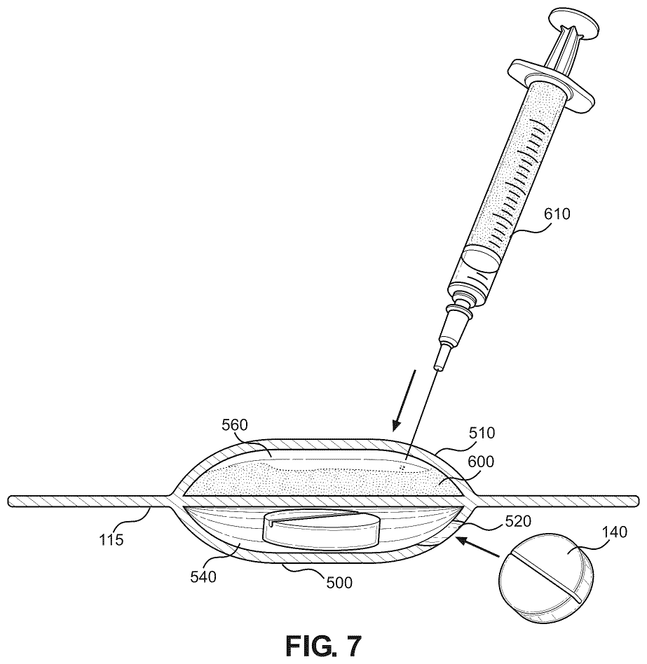

Referring now to FIG. 7, there is shown a perspective view of an alternate embodiment of the pet treat pocket in use. In one use, at least one pill 140 is inserted into the first pocket 500 via the opening 520. Once the proper dosage is placed within the first pocket 500, the opening 520 may be sealed closed by applying pressure to adhere the upper edge of the opening 520 to the top surface 115 of the flexible member. Additionally, the user may inject liquid medication 600 into the second pocket 510 via a syringe 610 until the desired dosage of liquid medication 600 is stored within the second interior volume 560. Once the correct dose has been injected, the user may mold the wall of the second pocket 510 together to seal the injection site closed to ensure the liquid medication does not exit the second pocket 510. Additionally, the user may further fold the lip about the first and second pockets 500, 510 to provide an additional layer of material over the first and second pockets 500, 510 to ensure that the contents thereof remain in the first and second interior volumes 540, 560, respectively. The user may then feed the pet treat pocket to the desired pet to administer the pill 140 and the liquid medication 600 to the animal.

For the liquid medication a barrier shell with an amorphous fibre-based core that immediately sorbs the injection of liquids and at least up to 60% of its weight.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A pet treat pocket, comprising:
a flexible member including one or more first pockets disposed centrally on a top surface of the flexible member, wherein the one or more first pockets includes an opening disposed along an upper edge of the one or more first pockets providing access to a first interior volume;
one or more second pockets disposed centrally on a lower surface of the flexible member,
wherein the one or more second pockets comprise a second interior volume defined between and fully enclosed by an upper wall and a plurality of sidewalls of the one or more second pockets and the flexible member;
wherein the one or more first pockets are sized to encapsulate a pill within the first interior volume of the one or more first pockets;
wherein an exterior pocket length of each of the one or more first pockets and the one or more second pockets are less than a length of the flexible member, and an exterior pocket width of each of the one or more first pockets and the one or more second pockets are less than a width of the flexible member such that the flexible member extends beyond the exterior dimensions of the one or more first pockets and the one or more second pockets, thereby forming a lip about an exterior of the one or more first pockets and the one or more second pockets;
wherein the lip surrounds a circumference of each of the one or more first pockets and the one or more second pockets;
wherein the flexible member, the one or more first pockets, and the one or more second pockets each are composed of an ingestible and pet-safe material; and
wherein the ingestible and pet-safe material is configured to adhere to itself, such that the one or more first pockets are selectively sealable to the flexible member upon application of force between the flexible member and the upper edge of the one or more first pockets.

2. The pet treat pocket of claim 1, wherein the flexible member is a first layer and the one or more first pockets and the one or more second pockets each comprise an additional layer disposed on the flexible member.

3. The pet treat pocket of claim 1, wherein the lip is sized to completely enrobe an entirety of each of the one or more first pockets and the one or more second pockets when the flexible member is molded over the one or more first pockets and the one or more second pockets.

4. The pet treat pocket of claim 1, wherein the ingestible and pet-safe material is made of a soft inner hygoscopic glycerin based with multiple hydroxyl groups humectant to attract and retain fluids.

5. The pet treat pocket of claim 1, wherein the ingestible and pet-safe material is malleable by hand.

6. The pet treat pocket of claim 1, wherein the ingestible and pet-safe material further comprises a flavoring additive.

7. The pet treat pocket of claim 1, wherein the ingestible and pet-safe material is selected from the group consisting of a gelatin-based material, montmorillonite clay and a binding material, or without calcium bentonite.

8. The pet treat pocket of claim 1, wherein the ingestible and pet-safe material is impermeable to odors emanating from each of the first interior volume and the second interior volume.

9. The pet treat pocket of claim 1, wherein the ingestible and pet-safe material emits a masking odor to overpower odors emanating from each of the first interior volume and the second interior volume.

10. The pet treat pocket of claim 1, wherein a plurality of pet treat pockets are affixed together defining an elongated strip having the one or more first pockets on a top surface of the elongated strip and the one or more second pockets on a lower surface of the elongated strip, wherein the one or more first pockets and the one or more second pockets are disposed along a longitudinal axis of the elongated strip such that the opening of each of the one or more first pockets are oriented substantially perpendicular to the longitudinal axis.

11. The pet treat pocket of claim 10, wherein at least one perforation is disposed transversely across the elongated strip between adjacent pockets of the pluralities of one or more first pockets and the one or more second pockets, wherein the at least one perforation is substantially parallel to the opening of each first pocket of the plurality of first pockets.

12. The pet treat pocket of claim 11, wherein the elongated strip is subdivided into seven segments, wherein each segment is defined between perforations such that each segment comprises a first pocket and a second pocket thereon.

13. The pet treat pocket of claim 1, wherein an exterior wall of one or more pockets comprises an arcuate sidewall wherein the depth of one or more pockets at a central portion of the exterior wall is greater than a depth at each lateral edge of the exterior wall.

14. The pet treat pocket of claim 1, wherein the upper wall of the one or more second pockets comprises an arcuate sidewall, wherein a depth of the one or more second pockets are greater at a central portion of the upper wall than at each lateral edge of the upper wall.

15. The pet treat pocket of claim 1, wherein the flexible member is selectively movable between a molded configuration and an unmolded configuration, wherein with the molded configuration the lip is sealed against an entirety of an exterior wall of the one or more first pockets defining at least two walls between an interior volume and a surrounding area.

16. The pet treat pocket of claim 1, wherein the one or more first pockets are affixed to the flexible member along a lower edge, a pair of lateral edges, and a portion of the upper edge of the one or more first pockets.

17. The pet treat pocket of claim 1, wherein the one or more first pockets comprises an identical cross-sectional area as the one or more second pockets, such that the first interior volume and the second interior volume are equal.

18. The pet treat pocket of claim 1, wherein the upper edge of the one or more first pockets further comprises an arcuate protrusion extending towards the flexible member, such that a height of the opening of the one or more first pockets are less than a distance between an exterior wall and the flexible member.

19. The pet treat pocket of claim 1, wherein a liquid medication is injected into the one or more second pockets via a syringe until a desired dosage of the liquid medication is stored within the second interior volume.

20. The pet treat pocket of claim 19, wherein the liquid medication is a barrier shell with an amorphous fibre-based core that immediately sorbs the injection of liquids and at least up to 60% of its weight.
